(19) United States
(12) Patent Application Publication
(10) Pub. No.: US 2006/0030891 A1
(43) Pub. Date: Feb. 9, 2006

(54) Method and apparatus for remote wireless communication with a medical device

(75) Inventors: William E. Saltzstein, Woodinville, WA (US); H. Ward Silver, Vashon, WA (US)

Correspondence Address: SHUMAKER & SIEFFERT, P. A., 8425 SEASONS PARKWAY, SUITE 105, ST. PAUL, MN, 55125, US

(73) Assignee: Medtronic Physio-Control Manufacturing Corp., Redmond, WA (US)

(21) Appl. No.: 11/247,515

(22) Filed: Oct. 11, 2005

Related U.S. Application Data
(62) Division of application No. 09/898,754, filed on Jul. 3, 2001.
Application No. 09/898,754 is a continuation-in-part of application No. 09/311,905, filed on May 14, 1999, now abandoned.

(51) Int. Cl. A61N 1/39; A61N 1/08

(52) U.S. Cl. 607/5; 607/32; 607/60

(57) ABSTRACT

A medical device is configured to support two-way communication to remotely monitor and manage medical device parameter status and software. A two-way communication module is incorporated into the medical device. A remote monitoring service is configured to regularly communicate with the medical device to initiate status assessments of medical device parameters and perform software updates. The medical device then sends a return message to the remote monitoring service using the two-way communication network. The return message can include the requested information, parameter values, or information about the software updates. This system can be advantageously used to efficiently monitor a large number of portable or mobile medical devices in a manner that is transparent to the users of the medical devices.

### RELATIONSHIP TO OTHER APPLICATIONS

This is a continuation-in-part of U.S. patent application Ser. No. 09/311,905, filed May 14, 1999. U.S. patent application Ser. No. 09/311,905 is specifically incorporated herein by reference.

### FIELD OF THE INVENTION

The present invention is related to communication systems for use with a medical device and, more particularly, to remote wireless communication systems for medical devices using a two-way communication channel.

### BACKGROUND INFORMATION

In general, medical device reliability is an essential function for any party relying on the availability and proper function of the medical device. Some medical devices, such as conventional, defibrillators, are typically operated by trained personnel and have been generally expensive to purchase and maintain. Because of the relative expense of these devices and the skill/experience level of the trained personnel operating these devices, such medical devices generally include a variety of diagnostic tools to ensure proper function of the medical device. For example, the medical device may include self-test diagnostic functions that detect and indicate when the device is not properly functioning. In addition, technicians may manually check the medical devices by verifying the device's diagnostics and/or conducting additional maintenance tests on a regular basis. While manual monitoring of the medical devices by a skilled technician allows for the verification of medical device functionality, such an embodiment is typically limited to situations in which a number of medical devices are located in a centralized location, such as a hospital. Accordingly, such an embodiment is generally unavailable, or prohibitively expensive, for situations in which only a small number of medical devices are maintained at one location, or where a number of medical devices are maintained in separate locations.

In yet other embodiments, a remote monitoring service can initiate medical device self-tests and software reconfigurations within the medical device. These conventional remote monitoring systems tend to use either wired connections (e.g., LAN or telephone service) or wireless systems such as cellular telephone or specialized proprietary RF systems to communicate with the medical device and initiate testing. FIG. 1 is a block diagram illustrative of a conventional medical device communication system 10 having a medical device 11, a communication network 12, and a remote monitoring service 13. As indicated by the dashed lines in FIG. 1, the medical device 11 can be located at the user's premises or at a field site (e.g., when paramedics in responding to an emergency use a portable external defibrillator). The communication network 12 may be a wired system such as, for example, a normal telephone system 15 or a local area network (LAN) 16. Alternatively, the communication network 12 may be a wireless system such as, for example, a cellular telephone system 17 or a private wireless communication system 18 dedicated to monitoring medical devices. Some of these conventional wireless systems require that a vehicle with receiving equipment drive by the customer's premises in order to communicate with the medical device 11. The medical device 11 includes an interface 19 for communicating with the remote monitoring service 13 through the communication system 12.

The conventional remote monitoring service 13 is generally configured to obtain status information from the medical device 11. However, the remote monitoring service 13 is typically limited to implementing specific functions dependent on the use state of the medical device 11. For example, when the medical device 11 is powered, but not in operation, the medical device can provide self-test, calibration, expiration or maintenance information to the remote monitoring system. When the medical device 11 is in operation, the remote monitoring service 13 can send control information or the medical device 11 can provide patient diagnostic information to the remote monitoring system 13.

In the above-described system, the remote monitoring system relies on use of more advanced medical device diagnostic tests to determine the device status. Accordingly, these remote monitoring systems require more advanced, and therefore more costly, medical devices having the advanced diagnostic features. Moreover, current conventional remote monitoring systems are limited to providing medical device configuration changes, and are not able to send actual software updates to the medical device.

Thus, there is a need for a system and device for remote wireless communication with a medical device to obtain a status assessment and to provide software updates.

### SUMMARY OF THE INVENTION

A medical device is configured to support two-way communication to a remote monitoring system to monitor and manage medical device parameter status and software. A two-way communication module is incorporated into the medical device. A remote monitoring service is configured to regularly communicate with the medical device to initiate status assessments of medical device parameters and to perform software updates. The medical device then sends a return message to the remote monitoring service using the two-way communication network. The return message can include the requested information, parameter values, or information about the software update. This system can be advantageously used to efficiently monitor a large number of portable or mobile medical devices in a manner that is transparent to the users of the medical devices.

In accordance with an aspect of the present invention, a communication system is provided including a medical device having a communication module, a two-way communication network and a remote monitoring service. The remote monitoring service is configured to send a communication to the medical device using the two-way communication network, the communication including an instruction for the medical device to perform a status assessment of at least one medical device parameter when the medical device is not in use. Additionally, in response to receiving the communication, the medical device is configured to obtain the requested information and send a return communication back to the remote monitoring service using the two-way communication network, the return communication including the requested information. In another embodiment of the invention, the communication includes software update information and the medical device is configured to update the software stored in the medical device in response to receiving the software update information.

In accordance with another aspect of the present invention, a method for a remote monitoring service to communicate with a medical device using a two-way communication network is provided. In accordance with the method, the remote monitoring service initiates a communication to the medical device using the two-way communication network, the communication including an instruction by the remote monitoring service for the medical device to perform a status assessment of at least one medical device parameter when the medical device is not in use. The medical device receives the communication and performs the status assessment of the at least one medical device parameter in response to receiving the communication. The medical device sends a return communication to the remote monitoring service using the two-way communication network, the message including the requested status assessment. In another embodiment of the present invention, the communication includes software update information to update software stored in the medical device and, upon receiving the communication, the medical device performs a software update with the software update information.

In accordance with yet another aspect of the present invention, a defibrillator is provided having a power source, a charging circuit coupled to the power source, an energy storage device coupled to the charging circuit, an output circuit coupled to the energy storage device, a pair of electrodes coupled to the output circuit, a two-way communication module, and a controller having a memory. The controller is coupled to the two-way communication module, the charging circuit and the output circuit. The controller is configured to selectively cause the charging circuit to transfer energy from the power source to the energy storage device and to cause the output circuit to transfer energy from the energy storage device to the electrodes. Additionally, the controller is further configured to operate the two-way communication module to (i) receive a communication from a remote monitoring service via a two-way communication network, the communication including an instruction for the medical device to perform a status assessment of at least one medical device parameter when the medical device is not in use, and (ii) send a communication to the remote monitoring service via the two-way communication network, the communication including the requested information. In another embodiment of the present invention, the communication includes software update information to update software stored in the controller memory and the controller is further configured to perform the software update with the software update information.

In still a further aspect of the present invention, a defibrillator is provided having a power source, a charging circuit coupled to the power source, an energy storage device coupled to the charging circuit, an output circuit coupled to the energy storage device, a pair of electrodes coupled to the output circuit, and a two-way communication module. The defibrillator also includes defibrillator control means for selectively causing the charging circuit to transfer energy from the power source to the energy storage device and to cause the output circuit to transfer energy from the energy storage device to the electrodes. Additionally, the defibrillator includes communication module control means, coupled to the two-way communication module, for receiving and processing a communication from a remote monitoring service via a two-way communication network, the communication including an instruction for the defibrillator to perform a status assessment of at least one defibrillator parameter when the defibrillator is not in use, and for sending a communication to the remote monitoring service via the two-way communication network, the communication including the requested information. In another embodiment of the present application, the communication includes software update information and the defibrillator control means further include means for updating defibrillator software with the software update information.

In still a further aspect of the present invention, a communication device for use in conjunction with a medical device, a two-way communication network and a remote monitoring service is provided. The communication device includes a controller and a two-way communication circuit coupled to the controller. In response to a communication from the remote monitoring service using the two-way communication network, the communication including an instruction for the medical device to obtain a status assessment of at least one medical device parameter when the medical device is not in use, the communication device is configured to obtain the requested information from the medical device and send a return communication back to the remote monitoring service using the two-way communication network, the return communication including the requested information. In another embodiment of the present invention, the communication includes software update information and the communication device is configured to instruct the medical device to perform a software update.

In yet another aspect of the present invention, a communication system including a medical device having a two-way communication module, a communication network and a remote monitoring service is provided. The remote monitoring service is configured to send a communication to the medical device via the communication network using a first communication medium, the communication including a request for information from the medical device. Additionally, in response to receiving a communication, the medical device is configured to obtain the requested information and send a return communication to the remote monitoring service via the communication network utilizing a second communication medium. The first and second communication media are different.

### BRIEF DESCRIPTION OF THE DRAWINGS

The foregoing aspects and many of the attendant advantages of this invention will become more readily appreciated by reference to the following detailed description, when taken in conjunction with the accompanying drawings listed below.

FIG. 1 is a block diagram illustrative of a conventional communication system for medical devices;

FIG. 2 is a block diagram illustrative of a remote wireless communication system for use with medical devices in accordance with the present invention;

FIG. 3 is a flow diagram illustrative of the operation of a remote wireless communication system in accordance with the present invention;

FIG. 4 is a block diagram illustrative of a defibrillator for use in a remote monitoring network in accordance with the present invention;

FIG. 5 is a block diagram illustrative of a remote monitoring service for use in a remote monitoring network in accordance with the present invention;

FIG. 6 is a flow diagram illustrative of the operation of a remote wireless communication system in accordance with the present invention;

FIG. 7 is a block diagram illustrative of a medical device configured to provide information regarding the location of the medical device to a remote monitoring server in accordance with the present invention;

FIG. 8 is a block diagram illustrative of a remote wireless and data network communication system for use with medical devices in accordance with the present invention;

FIGS. 9 and 10 are block diagrams illustrative of data network communication links between a remote monitoring service and a two-way communication network in accordance with the present invention;

FIG. 11 is a block diagram illustrative of a remote wireless communication system for use with medical devices in accordance with the present invention; and

FIG. 12 is a block diagram illustrative of a remote communication system for use with independent subsystems of medical devices in accordance with the present invention.

### DETAILED DESCRIPTION

FIG. 2 is a simplified diagram illustrative of a remote wireless communication system 20 for use with medical devices in accordance with the present invention. The communication system 20 includes a medical device 21 having a two-way communication module 22, a two-way communication network 23 and a remote monitoring service 24. In an illustrative embodiment of the present invention, the communication network 23 utilizes any one of a variety of communication media and/or communication methods to transfer data. Examples of suitable communication media and/or communication methods include, but are not limited to, wired communication media/methods such as public switched telephone networks (“PSTN”), wired digital data networks, such as the Internet or a local area network (“LAN”), co-axial cable, fiber optic cable and the like. Examples of suitable wireless communication media and/or communication methods include, but are not limited to, wireless telephony (“cellular”) including analog cellular, digital personal communications service (“PCS”), short message service (“SMS”), and wireless application protocol (“WAP”). Other suitable wireless communication media/methods include wireless digital data networks, such as 802.11 wireless LAN (“WLAN”), two-way paging networks, specialized mobile radio systems, infrared, and non-licensed ISM-service communication links, such as Bluetooth. Further, communication methods, either wireless or wired, include Internet protocol (“IP”) addressing. One skilled in the relevant art will appreciate that additional or alternative communication media/methods may be practiced with and are considered within the scope of the present invention. In an actual embodiment of the present invention, the communication network utilizes two or more communication media/methods to communicate with the medical device 21 and/or the remote monitoring network 24.

Although only one medical device 21 is shown in FIG. 2, it will be appreciated that the remote monitoring service 24 can monitor a relatively large number of medical devices (not shown) using the two-way communication network 23 of the communication system 20. The medical device 21 communicates with the remote monitoring service 24 through the two-way communication network 23 by utilizing the two-way communication module 22. In an illustrative embodiment of the present invention, the two-way communication module 22 includes components corresponding to the specific communication medium/method utilized by the communication network 23. Alternatively, the communication module 22 may include multiple components to allow the medical device 21 to utilize a number of communication media/methods. One skilled in the relevant art will appreciate that a communication module 22 conforming to a specific communication medium and communication method of a communication network 23 will include a number of components necessary to interact with the communication network. However, because the necessary components for each medium/method can be found as original equipment manufacturing units or within an adopted set of specifications, details of acceptable components for each potential communication medium/method will not be described in further detail herein.

As illustrated in FIG. 2, in an illustrative embodiment of the present invention, the medical device 21 uses the two-way communication module 22 to interface with the communication network 23 through a wireless channel, as indicated by an arrow 25. In addition, the two-way communication network 23 communicates with the remote monitoring service 24 through a channel (wired or wireless), indicated by an arrow 26. In an illustrative embodiment, the channel 26 is a standard telephone connection with standard modem interfaces 27 and 28 in remote monitoring service 24 and two-way communication network 23, respectively. One skilled in the relevant art will appreciate that the communication network 23 may utilize a first communication medium/method to communicate with the medical device 21 and a second communication medium/method to communicate with the remote monitoring service 24

FIG. 3 is a flow diagram illustrative of the operation of the remote wireless communication system 20 (FIG. 2) in accordance with the present invention. Referring to FIGS. 2 and 3, the remote wireless communication system 20 operates as follows. In a block 31, the remote monitoring service 24 initiates a communication addressed to the desired medical device (e.g., the medical device 12 in this example). The communication includes, for example, instructions (or codes representing instructions) for the medical device 21 to provide status assessment, perform self-tests, change the configuration, and/or update the software of the medical device 21. For example, the status assessment of a medical device can include information such as power supply levels, software versions, configuration settings, counters indicating a number of times the unit has been utilized, and the like.

The remote monitoring service 24 initiates the communication by sending a request to the two-way communication network 23 with the desired medical device 21 and data. In general, any wired or wireless data transmission network may be used to initiate the communication. For example, in an illustrative embodiment of the present invention, the monitoring service 24 initiates this communication by calling a “pager” number previously assigned to the medical device 21, using a standard telephone line and modem connection. In another illustrative embodiment of the present invention, the monitoring service 24 initiates this communication by specifying an IP address previously assigned to the medical device 21, or group of medical devices.

In an actual embodiment of the present invention, the remote monitoring service 24 encodes instructions to be transferred by the communication network 23. The encoding of the instructions may include causing medical device instructions to conform to a specific format required by the medical device and/or providing data encryption to the instructions. Further, error correction techniques are often used to ensure correct reception of the encoded instructions in the presence of impairments in the transmission channel. The two-way communication network 23 includes a switch (not shown) that accepts the communication from the remote monitoring service 24 and generates the desired instruction in accordance with the communication medium/method utilized by the particular medical device 21.

At block 33, the medical device 21 receives the communication via the communication module 22 and then extracts the instructions from the communication. The communication module 22 may be configured to send an acknowledgment communication back to the two-way communication network 23 to confirm receipt of the communication. At block 35, the medical device 21 performs the extracted instructions. For example, the medical device 21 may perform a status assessment of a predetermined number of medical device parameters when the device is not in use, or it may perform a status assessment of one or more medical device parameters specified by the remote monitoring service 24 in the communication.

At block 37, the medical device 21 transmits a communication back to the remote monitoring service 24 through the two-way communication network 23. For example, in accordance with communication network 23 utilizing a two-way paging protocol, a “return page” is transmitted in a return channel that is different from the channel (i.e., frequency band) used to transmit the page initiated by the remote paging service 24 in block 31. This type of system allows the medical device 21 to initiate transmission of the return communication without having to wait to be polled by the two-way communication network 23.

In an actual embodiment of the present invention, the return communication contains self-test results, device parameter data, or condition data, depending on the nature of the instructions sent by the remote monitoring service 24. The return communication from the medical device 21 can be immediately provided by the two-way communication network 23 to the remote monitoring service 24 via the same data transmission network that the remote monitoring service 24 used to initiate the communication. For example, two-way communication network 23 may maintain an open wireless communication channel to obtain the return communication from the medical device 21. Alternatively, the return communication may be stored locally by the medical device 21 for later transmission or may be stored within the two-way communication network 23 in a “mail box” for later retrieval by the remote monitoring service 24.

At block 39, the remote monitoring service 24 extracts the information from the return communication. This information can then be displayed for analysis by a technician at the remote monitoring service 24. Alternatively, the remote monitoring service 24 may be configured with a computer programmed to analyze the information. The remote monitoring service 24 can then alert a technician or even the customer when analysis of the information indicates a problem. Additionally, the remote monitoring service 24 may initiate a request for a replacement medical device 21 upon detection of any abnormal status assessment.

One skilled in the relevant art will appreciate that the present invention can include a remote communication system for medical devices in which several medical devices are monitored by the remote monitoring service 24 using the two-way communication network 23. The remote monitoring service 24 would be configured to “poll” each medical device at given intervals, such as at least once a day, depending on the number of medical devices and the capacity of the two-way communication network 23.

FIG. 4 is a block diagram illustrative of a defibrillator medical device 21 for use in the remote monitoring network 20 in accordance with the present invention. Although a defibrillator is illustrated in this embodiment, those skilled in the art will be able to implement other embodiments using other types of medical equipment, without undue experimentation. In addition to the two-way communication module 22, the defibrillator 21 includes a controller 40, a power source 41, an energy storage device 42, an output circuit 43 and output electrodes 44 and 45. To facilitate understanding of the invention, the same reference number may be used in several drawings to indicate elements having the same or similar structure or function. In this embodiment, the two-way communication module 22 is implemen ted as a two-way paging module with a CreataLink2™ two-way paging module, available from Motorola, Inc. Schaumburg, Ill. This embodiment uses the ReFLEX™ two-way paging protocol. Of course, in other embodiments, other suitable communication modules, including original equipment manufacturer (“OEM”), two-way communication modules and/or communication protocols may be utilized.

In addition, the controller 40 includes a microprocessor (not shown) such as, for example, a model MC68332 available from Motorola, along with a memory 46. Preferably, the memory 46 includes random access memory such as a DRAM (dynamic random access memory) or SRAM (static random access memory), and non-volatile memory such as an EEPROM (electrically erasable programmable read only memory). The EEPROM can be used to store software programs executed by the processor (not shown). In addition, the EEPROM allows the stored software programs to be remotely updated. The power source 41 is implemented with a battery, such as a LP500 battery available from Medtronic Physio-Control Manufacturing Corp., Redmond, Wash. The energy storage device 42 is implemented with a capacitor with a capacitance of about 190-200 μF. The output circuit 43 is implemented in an H-bridge configuration, which facilitates generating biphasic output pulses. For example, the output circuit 43 can be implemented as disclosed in U.S. patent application Ser. No. 08/811,833 filed Mar. 5, 1997, entitled “H-Bridge Circuit For Generating A High-Energy Biphasic Waveform In An External Defibrillator” by J. L. Sullivan et al. In one embodiment, the controller 40, the power source 41, the energy storage device 42, the output circuit 43 and the electrodes 44 and 45 are the same as used in a LP500 AED available from Medtronic Physio-Control Manufacturing Corp. That is, the hardware aspect of medical device 21 is basically equivalent to a LP500 AED with the addition of the two-way pager module 22, along with suitable software programming stored in the memory 46.

FIG. 5 is a block diagram illustrative of a remote monitoring service 24 for use in a remote monitoring network 20 in accordance with the present invention. In an illustrative embodiment, the remote monitoring service 24 includes a modem 51, a control unit 52 and a user interface 53 having a display. The modem 51 is part of interface 27 (FIG. 2) and is implemented with a standard commercially available modem. The control unit 52 is connected to the modem 51 and includes a standard processor and associated memory (not shown). As an alternate embodiment of the present invention, the modem 51 may be omitted or replaced by an interface to a communications system. The control unit 52 is programmed to initiate pages to be transmitted through the two-way communication network 23 (FIG. 2), as described above in conjunction with FIGS. 2 and 3. For example, the control unit 52 may be programmed to initiate status assessment communications to be sent to the medical device 21 (FIG. 2) according to a programmed schedule. In particular, the control unit 52 would send a communication request to the two-way communication network 23 (FIG. 2) through the modem 51 as previously described, which would then transmit the requested communication to the medical device 21. In addition, the control unit 52 is programmed to process return communications transmitted by the medical device 21 (FIG. 2). The control unit 52 further may be programmed to encode any communications sent to one or more medical devices.

In an illustrative embodiment of the present invention, the control unit 52 is also connected to the user interface 53. This feature can be used to display the processed return communications from a medical device 21 so that a user (not shown) can view the information contained in the received return communication. The user can then analyze the displayed information and take appropriate action. For example, the return communication may contain the results of a status assessment initiated by the remote monitoring service 24. Accordingly, the user interface 53 displays values corresponding to the medical device parameters assessed and the user can then contact the customer (or the party responsible for the medical device 21) to perform maintenance, such as taking the medical device 21 to a repair facility.

The user may also use the user interface 53 to initiate communications to the medical device 21. For example, this feature may be used to send a medical device software update or medical device reconfiguration information to the medical device 21. One skilled in the relevant art will appreciate that a medical device 21 includes software, or other logical controls, that controls the various medical device hardware components. Moreover, to allow the medical device 21 to incorporate multiple functions or to adjust the function of the medical device, some medical devices also include medical device configuration information, or device protocol information, that allows one or more parameters modifications within the scope of the underlying medical device software. For example, defibrillator software generally controls the hardware components utilized to produce a therapy signal, while the defibrillator configuration data can adjust the parameters of the hardware components to vary the function of the therapy signal. Other configuration information can include communication protocols or display protocols that do not affect the underlying medical device software. To allow flexibility in the function of the medical device, the present invention allows the medical device configurations to be changed, or modified, without having to modify the underlying medical device software. Accordingly, the remote monitoring service 24 implements medical device configuration changes by transmitting configuration information to the medical device 21.

The present invention further allows the designation, through the user interface 53, of medical device software update data to be transmitted to the medical device. Upon obtaining the medical device software update, the previous software may be discarded, saved or merely modified. Accordingly, if the medical device 21 obtains configuration information from the remote monitoring service, the medical device 21 implements the configuration change and sends a return communication via the communication network on the success of the configuration change. Similarly, if the medical device obtains software update information, the medical device implements the software update and sends a return communication via the communication network on the success of the software update.

One skilled in the relevant art will appreciate that the user interface 53 may utilize any one of a variety of display techniques, including, but not limited to graphical user interfaces, textual displays and/or animation. Moreover, a communication from the remote monitoring service 24 may include a combination of configuration and software update information.

FIG. 6 is a flow diagram illustrative of the operation of the remote wireless communication system 20 (FIG. 2), according to another embodiment of the present invention. This embodiment utilizes the ability of some communication protocols to support independent transmission of a return communication to send a message to the remote monitoring service 24. That is, the medical device 21 can send a communication to the remote monitoring service 24 without first having received an instruction from the remote monitoring service 24. With reference to FIG. 6, at block 61, the medical device 21, in a self-initiated process, initiates some sort of medical device action. For example, the medical device 21 may be an AED having a control unit (e.g., the controller 40 in FIG. 4) that constantly monitors a variety of medical device parameters, such as the voltage of its battery (e.g., the power source 41 in FIG. 4).

In one embodiment of the present invention, if the battery voltage drops below a predetermined threshold level, the AED will automatically initiate a communication via the two-way communication network 23 to the remote monitoring service 24. In another embodiment of the present invention illustrating a status assessment, the AED may initiate a communication to the remote monitoring service 24 to report values for various medical device parameters, such as the battery voltage, to provide a status assessment. In this embodiment, the status assessment communication may be initiated if the medical device 21 detects a change in the parameter value or if a sufficient amount of time has elapsed since the last report of the parameter value.

In accordance with the present invention, some medical devices may include an on-board controller and clock system such that the controller can be programmed to periodically initiate various medical device actions such as self-tests, status assessments, and the like. Alternatively, the medical device 21 may initiate a communication with the remote monitoring service 24 upon being activated by a user or after being used. For example, the medical device 21 may be programmed to initiate communications indicating when the medical device 21 is used, if the medical device 21 fails in an attempted use, or to communicate the status of the device after it has been utilized.

At block 63, the medical device 21 initiates a communication through two-way communication network 23 to provide the medical device information to the remote monitoring service 24. Block 63 is performed in essentially the same manner as block 37 (FIG. 3), except that in block 63, the return communication is not in response to an instruction sent by the remote monitoring service 24. That is, the information is generated by the medical device 21 and sent via the two-way communication network 23 to the remote monitoring service 24 without prompting by the remote monitoring service 24. This embodiment can be used to reduce the processing load on the control unit 52 (FIG. 5) of the remote monitoring service 24 when a large number of medical devices are being monitored. Blocks 65 and 67 are then performed by the remote monitoring service 24 in essentially the same manner as blocks 37 and 39 (FIG. 3), described above.

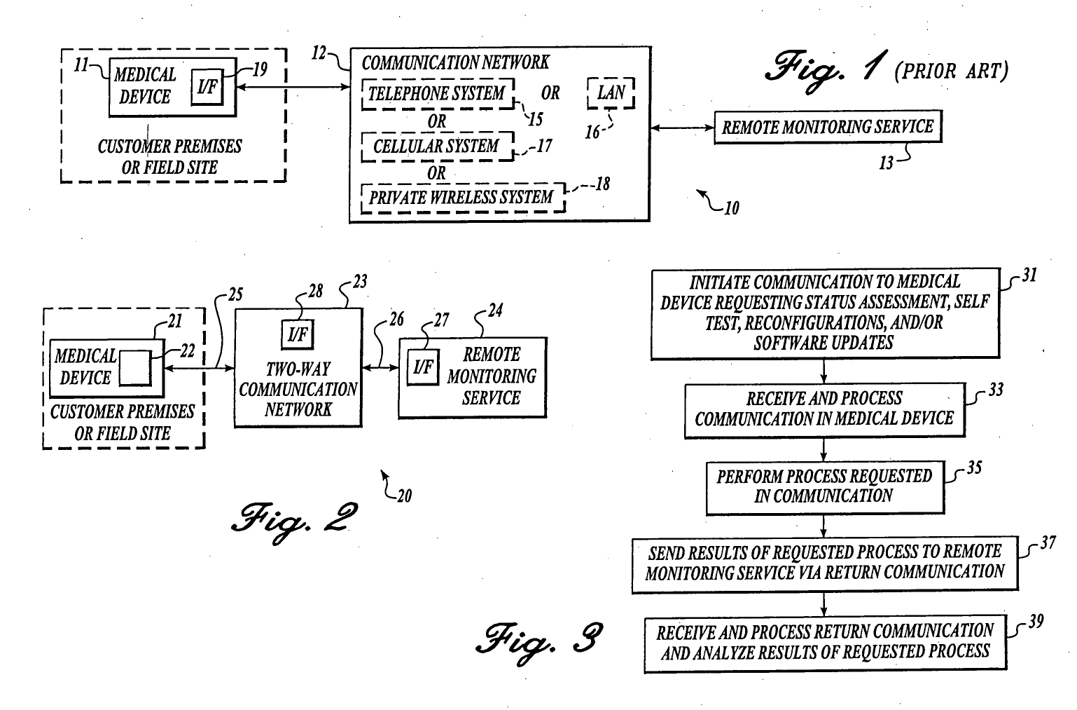

FIG. 7 is a block diagram illustrative of a medical device 70 configured to provide information regarding the location of the medical device 70 to the remote monitoring service 24 (FIG. 2), according to one embodiment of the present invention. In this embodiment, the medical device 70 includes the two-way communication module 22 (as described above in conjunction with FIG. 2), a location or navigation subsystem (such as a GPS (global positioning system)) module 71 and a controller 73. In addition, the medical device 70 includes the normal medical device circuitry 75 that the medical device 70 would have to perform its intended medical functions. For example, if the medical device 70 were an AED, the medical device circuitry 75 would typically include the power source 41, the energy storage device 42, the output circuit 43 and the electrodes 44 and 45 that an AED described above in conjunction with FIG. 4.

In one embodiment, the controller 73 is the same as the controller 40 (FIG. 4) with additional software programming to interact with the location or navigation subsystem 71. The location or navigation subsystem 71 is implemented with a standard location or navigation subsystem such as, for example, a suitable module of the SiRFstar I/LX Product Family, available from SiRF Technology, Inc., Santa Clara, Calif. The location or navigation subsystem 71 is used to detect the location of the medical device 70 in the standard manner. In particular, the controller 73 is programmed to query the location or navigation subsystem 71 to provide the current location of the medical device 70, which the controller 73 then causes to be transmitted to the remote monitoring service 24 (FIG. 2) using the two-way communication module 22. The medical device 70 can provide its current location in response to a request from the remote monitoring service 24 as described above in conjunction with FIG. 3, or on its own as described above in conjunction with FIG. 6. This feature can be used to track the location of portable medical devices such as AEDs.

FIG. 8 is a block diagram illustrative of a remote wireless and data network communication system 80 for use with medical devices according to an actual embodiment of the present invention. Specifically, the remote wireless and data network communication system 80 includes a data network point-of-presence (“POP”) interface and the channel 26 (FIG. 2) includes a data network interface 81. For example, the data network and data network POP may be implemented using the Internet and a Web site. In this embodiment, the remote monitoring service 24 is configured to send communication requests to remote medical devices (not shown) through the data network POP of the two-way communication network 23, using the data network interface 81. The data network interface 81 is configured to follow the procedures defined by the two-way communication network 23 for sending communications using its data network POP. In response to data network POP-based communication requests from the remote monitoring service 24, the two-way communication network 23 sends out communications to the addressed remote medical devices in the standard manner. The remote medical devices then send return communications as described above. The return communications from the remote medical devices are processed by the two-way communication network 23, which then provides the communication from the remote medical devices to the remote monitoring service 24. As previously described, the two-way communication network 23 may provide the return communication via one of the communication media/methods as previously described. Alternatively, the return communication may be in the form of data network email.

In an alternative embodiment, the two-way communication network 23 may also support direct data network email communication requests. This alternative embodiment is similar to the embodiment of FIG. 8, except that the remote monitoring service 24 is configured to send communication requests to the two-way communication network 23 by Internet email rather than through accessing the data network POP of the two-way communication network 23.

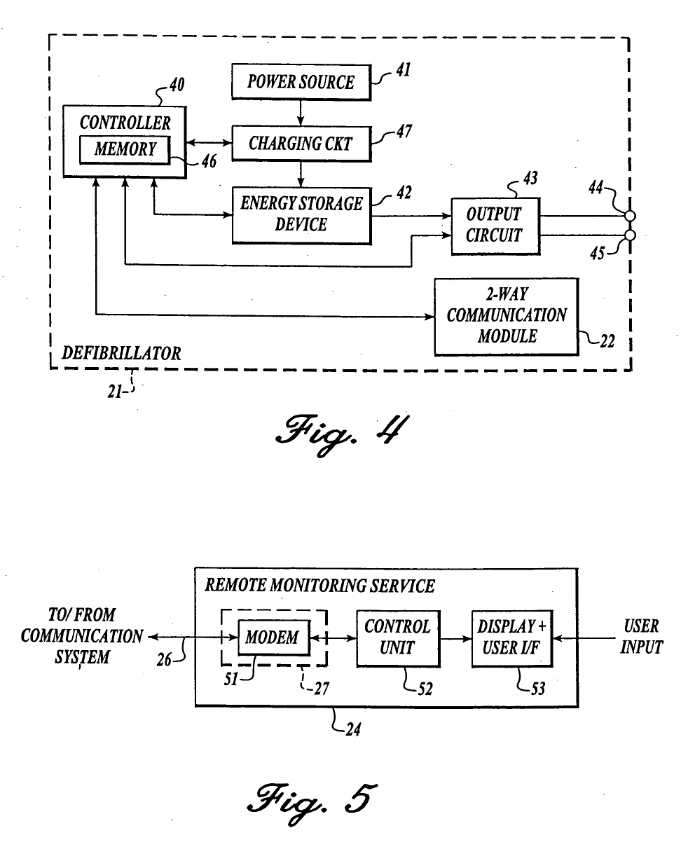

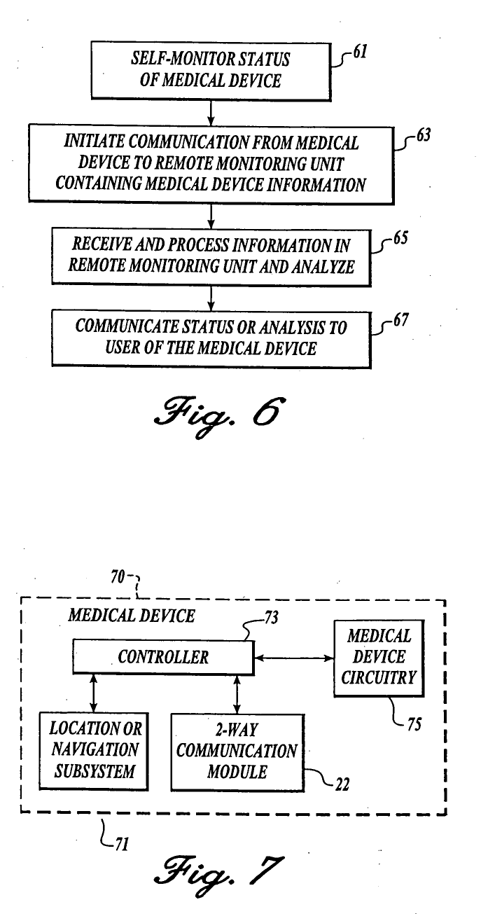

FIG. 9 illustrates the data network POP interface of the remote monitoring service 24 (FIG. 8), according to one embodiment of the present invention. In this embodiment, the remote monitoring service 24 accesses the data network through a data network access provider 90. The remote monitoring service 24 includes an interface 91 for establishing a connection with the data network access provider 90 over a line 92. The interface 91 can be a standard modem and the line 92 can be a standard telephone network. One skilled in the relevant art will understand that the remote monitoring service 24 may connect to the data network access provider 90 utilizing any one of a variety of communication media/methods.

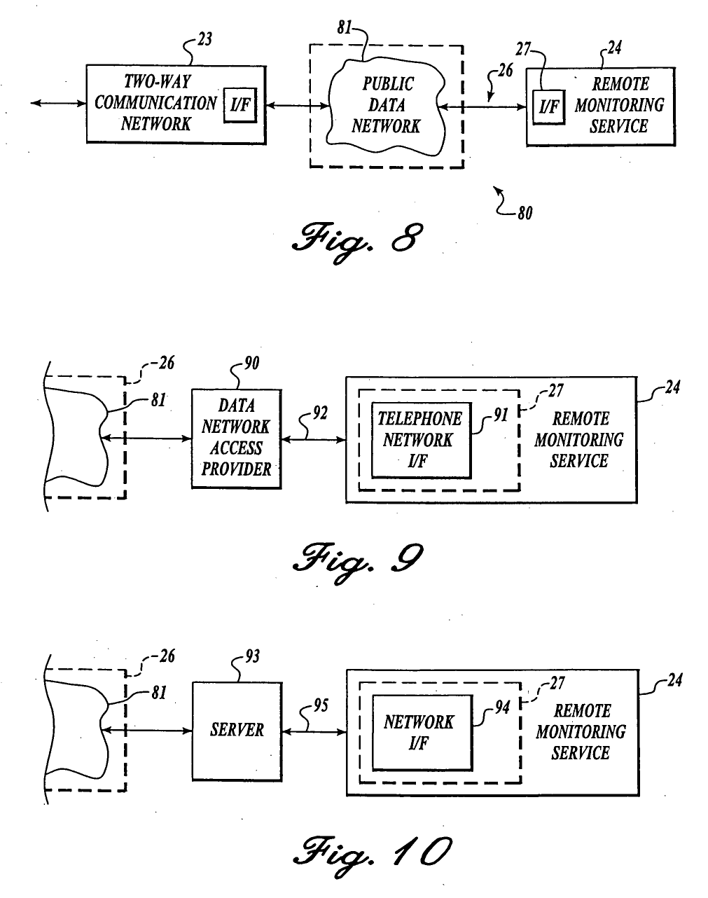

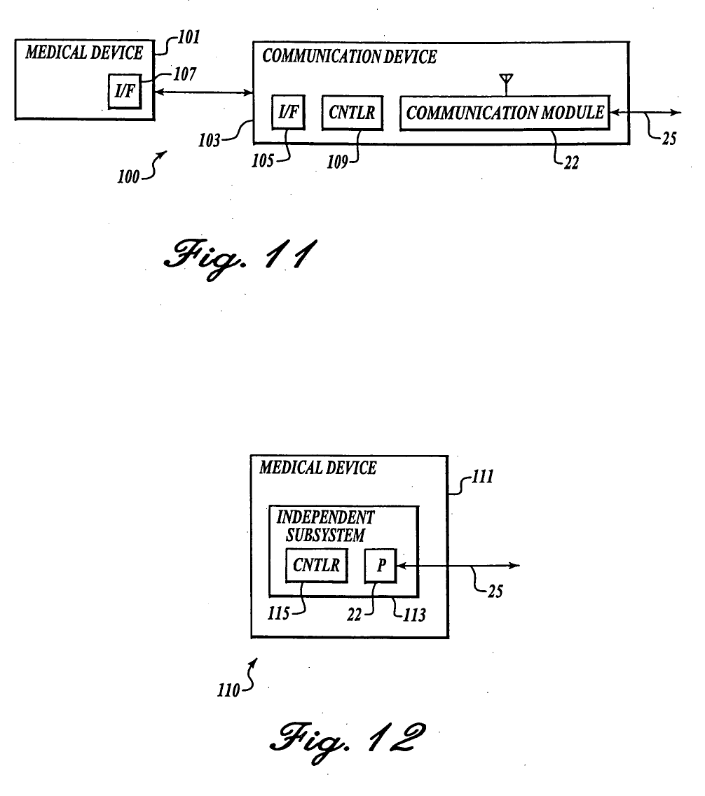

FIG. 10 illustrates the Internet interface of the remote monitoring service 24 (FIG. 8), according another embodiment of the present invention. This embodiment is similar to the embodiment of FIG. 9, except that the remote monitoring service uses a direct data network connection instead of a data network access provider 90. In this embodiment, the remote monitoring service 24 is connected to the data network by a server 93. The remote monitoring service 24 is connected to the server 93 via a line 95. The line 95 can be any suitable connection such as, for example, a direct cable connection, or an internal or Intranet network connection such as an Ethernet connection. Such direct data network connections are well known using commercially available equipment and software.

FIG. 11 illustrates part of a remote wireless communication system 100 for use with medical devices according to another illustrative embodiment of the present invention. The system 100 includes a conventional medical device 101 and a separate communication device 103. The rest of the system is as described in conjunction with FIG. 2, with the medical device 101 and the communication device 103 replacing the medical device 21. The communication device 103 and the medical device 101 include an interface 105 and an interface 107, respectively, for supporting communication between the devices. In one embodiment, the interfaces 105 and 107 form an RS-232-C bus connection.

The communication device 103 includes a communication module 22 as described above in conjunction with FIG. 2. The communication device 103 has a controller 109 and is configured to receive communications sent by the remote monitoring service 24 and pass instructions within the received communications to the medical device 101. In addition, the communication device 103 is configured to download data (e.g., self-test data) from the medical device 101 (either in response to the page from the remote monitoring service 24 or self-initiated) and send a return communication with the downloaded data to the remote monitoring service 24. Additionally, the medical device 101 can also include a communication module 22, as illustrated and described in relation to FIG. 2, that works in conjunction with communication device 103, or serves as a backup communication device.

FIG. 12 illustrates part of a remote wireless communication system 110 for use with medical devices, according to another embodiment of the present invention. The system 110 includes a medical device 111 having some type of embedded logic to process data and make local control decisions, generally referred to as an independent subsystem 113. The rest of the system 110 is as described above in conjunction with FIG. 2, with the medical device 111 replacing medical device 21.

The independent subsystem 113 can be an independent battery similar to that disclosed in U.S. patent application entitled “Smart Battery With Maintenance And Testing Functions, Communications, And Display”, Ser. No. 09/237,193 filed on Jan. 26, 1999, which is assigned to the same assignee as the present invention, except that the independent subsystem 113 includes communication module 22. The independent subsystem 113 includes a controller 115 that is similar to the controller 73 described above in conjunction with FIG. 7, which allows the independent subsystem 113 to be configured to inter-operate with the communication module 22 as previously described. One skilled in the relevant art will appreciate that the amount of embedded logic within the independent subsystem 113 may vary with the processing requirements of the medical device 111 and/or the resources (e.g., power, memory, processing) available to the independent subsystem.

The embodiments of the remote wireless communication system described above are illustrative of the principles of the present invention and are not intended to limit the invention to the particular embodiments described. For example, in light of the present disclosure, those skilled in the art can adapt the two-way communication system to medical devices other than AEDs without undue experimentation. In addition, those skilled in the art can adapt the two-way communication system to use other wired or wireless public data network interfaces or wireless telephone interfaces in other embodiments. Accordingly, while the preferred embodiment of the invention has been illustrated and described, it will be appreciated that various changes can be made therein without departing from the spirit and scope of the invention.

1-9. (canceled)

10. A communication system comprising:
a medical device having a communication module;
a two-way communication network; and
a remote monitoring service,
wherein the remote monitoring service is configured to send a communication to the medical device using the two-way communication network, the communication including software update information to update software stored in the medical device, and wherein, in response to receiving the communication, the medical device is configured to update the software stored in the medical device in response to receiving the software update information.

11. The system as recited claim 10, wherein the medical device is a portable defibrillator.

12. The system as recited in claim 10, wherein the two-way communication network includes a wireless communication network.

13. The system as recited in claim 10, wherein the two-way communication network includes a wired communication network.

14. The system as recited in claim 10, wherein the medical device is further configured to send a return communication once the software update has been complete.

15-20. (canceled)

21. A method for a remote monitoring service to communicate with a medical device having software using a two-way communication network, the method comprising:
initiating a communication from the remote monitoring service to the medical device using the two-way communication network, the communication including software update information to update software stored in the medical device;
receiving the communication in the medical device;
performing a software update with the software update information in response to receiving the communication in the medical device.

22. The method as recited in claim 21 further comprising sending a return communication from the medical device to the remote monitoring service utilizing the two-way communication network, wherein the return communication includes information about the software update has been completed.

23. The method as recited in claim 21, wherein the medical device is a portable defibrillator.

24. The method as recited in claim 21, wherein the two-way communication network includes a wireless communication network.

25. The method as recited in claim 24, wherein the two-way communication wireless network includes an analog cellular telephony network.

26. The method as recited in claim 21, wherein the two-way communication network includes a two-way paging network.

27. The method as recited in claim 21, wherein the two-way communication network includes a specialized mobile radio network.

28-33. (canceled)

34. A defibrillator comprising:
a power source;
a charging circuit coupled to the power source;
an energy storage device coupled to the charging circuit;
an output circuit coupled to the energy storage device;
a pair of electrodes coupled to the output circuit;
a two-way communication module; and
a controller having a memory coupled to the two-way communication module, charging circuit and the output circuit, wherein the controller is configured to selectively cause the charging circuit to transfer energy from the power source to the energy storage device and to cause the output circuit to transfer energy from the energy storage device to the electrodes, and
wherein the controller is further configured to operate the two-way communication module to receive a communication from a remote monitoring service via a two-way communication network, the communication including software update information to update software stored in the controller memory, and
wherein the controller is further configured to perform the software update with the software update information in the communication.

35. The defibrillator as recited in claim 34, wherein the controller is further configured to operate the two-way communication module to send information about the software update.

36. The defibrillator as recited in claim 34, wherein the two-way communication network includes a wireless communication network.

37. The defibrillator as recited in claim 34, wherein the two-way communication network includes a wired communication network.

38-42. (canceled)

43. A defibrillator comprising:
a power source;
a charging circuit coupled to the power source;
an energy storage device coupled to the charging circuit;
an output circuit coupled to the energy storage device;
a pair of electrodes coupled to the output circuit;
a two-way communication module;
defibrillator control means for selectively causing the charging circuit to transfer energy from the power source to the energy storage device and to cause the output circuit to transfer energy from the energy storage device to the electrodes; and
communication module control means, coupled to the two-way communication module, for receiving and processing a communication from a remote monitoring service via a two-way communication network, the communication including software update information,
wherein the defibrillator control means further includes means for updating defibrillator software with the software update information included in the communication.

44. The defibrillator as recited in claim 43, wherein the communication module control means further includes means for sending a communication to the remote monitoring service via the two-way communication network, the communication including information about the updating of the defibrillator software.

45. The defibrillator as recited in claim 43, wherein a processor and a memory are used to implement the defibrillator control means and communication module control means.

46. The defibrillator as recited in claim 43, wherein the two-way communication network includes a wireless communication network.

47. The defibrillator as recited in claim 43, wherein the two-way communication network includes a wired network.

48-51. (canceled)

52. A communication device for use in conjunction with a medical device having software, a two-way communication network and a remote monitoring service, the communication device comprising:
a controller; and
a two-way communication circuit coupled to the controller,
wherein in response to a communication from the remote monitoring service using the two-way communication network, the communication including software update information for the medical device, the communication device is configured to instruct the medical device to perform a software update.

53. The communication device as recited claim 52, wherein the communication circuit is in a separate unit external to the medical device, the separate unit being configured to be selectively coupled to the medical device.

54. The communication device as recited in claim 52, wherein the communication device is further configured to send a return communication from the medical device about the software update.

55. The communication device as recited in claim 52, wherein the medical device is a defibrillator.

56. The communication device as recited in claim 52, wherein the two-way communication network includes a wireless communication network.

57. The communication device as recited in claim 52, wherein the two-way communication network includes a wired communication network.

58-73. (canceled)
